(19) United States
(12) Patent Application Publication
(10) Pub. No.: US 2026/0232733 A1
(43) Pub. Date: Aug. 13, 2026

(54) METHOD FOR THYMUS REGENERATION

(71) Applicant: SMART IMMUNE, Paris (FR)

(72) Inventors: Olivier Negre, Paris (FR); Tayebeh-Shabi Soheili, Paris (FR); Hanem Sadek, Paris (FR)

(21) Appl. No.: 19/156,974

(22) PCT Filed: Feb. 22, 2024
(86) PCT No.: PCT/EP2024/054563

§ 371 Date: Aug. 15, 2025

Related U.S. Application Data
(60) Provisional application No. 63/486,327, filed on Feb. 22, 2023.

(51) Int. Cl. A61K 35/17; A61K 40/11; A61K 40/31; A61K 40/32; A61K 40/50; A61P 37/04; C07K 14/705; C12N 5/0783

(52) U.S. Cl. CPC A61K 35/17; A61K 40/11; A61K 40/31; A61K 40/32; A61K 40/50; A61P 37/04; C07K 14/7051; C12N 5/0636; C12N 2501/25; C12N 2501/42; C12N 2510/00; C12N 2533/52

(57) ABSTRACT

The present invention relates to methods of treating aging, and in particular immunoaging, of restoring thymus function and of regenerating the thymus in subjects in need thereof, comprising the administration of T cell progenitors comprising CD7+CD34− cells.

### FIELD OF INVENTION

The present invention relates to the field of treatment, in particular of therapeutic treatment, of aging, including immunoaging. In particular, the present invention relates to a method for restoring thymic function in a subject, thereby restoring polyclonal T cells production by the thymus of the subject.

### BACKGROUND OF INVENTION

Thymic hypoplasia secondary to aging causes a restricted T-cell repertoire and consequently impaired immunity. Of note, thymic involution mostly reckons on the thymic epithelium shrinkage. Enhancing intra-thymic T cell development and selection are therefore critical to replenishment of the peripheral T-cell compartment.

Previously, a clinical grade, feeder-free platform to manufacture ex-vivo human early T cell precursors was disclosed, based on the use of immobilized-Delta-like ligand 4 (DLL4) and human cytokines to enable the expansion and differentiation of CD34+ hematopoietic stem and progenitor cells (HSPC) into Human T Lymphoid progenitors (HTLP, also called T cell progenitors).

In the present invention, the Applicant demonstrates that these early T cell precursors, when administered to a subject with immunoaging, induce thymus regeneration and restore thymic function, in particular restore the capacity of the thymus to produce polyclonal T cells.

### SUMMARY

The present invention relates to a method of treating aging, and in particular immunoaging, in a subject, comprising the administration of T cell progenitors that comprise CD7+CD34− cells to the subject.

The present invention also relates to a method of restoring thymus function in a subject, comprising the administration of CD7+CD34− T cell progenitors to the subject.

The present invention further relates to a method of regenerating the thymus of a subject.

In some embodiments, the method of the invention is for restoring the polyclonal T cell production by the thymus of the subject.

In some embodiments, the subject is a human.

In some embodiments, the subject is of at least about 50 years in age, preferably of at least of about 60, 70, 80 or 90 years in age.

In some embodiments, the subject is affected or diagnosed with thymic atrophy or thymic involution.

In some embodiments, the CD7+CD34− cells comprise cells that are CD5− and/or CD1a−, preferably wherein at least about 50%, or at least about 60, 70, 80, 90 or 95% of the CD7+CD34− cells are CD5− and/or CD1a−.

In some embodiments, the T cell progenitors are genetically modified, such as, for example, to express an exogenous protein.

In some embodiments, the T cell progenitors are transfected or transduced with an exogenous nucleic acid encoding a Chimeric Antigen Receptor (CAR) or a recombinant TCR.

In some embodiments, the T cell progenitors express a cytokine, such as, for example, RANKL or Lymphotoxin A (LTA).

In some embodiments, the T cell progenitors are prepared by culturing CD34+ cells in the presence of an immobilized Notch ligand or fragment thereof, preferably wherein the Notch ligand or fragment thereof is immobilized on the inner surface of the culture vessel or on beads.

In some embodiments, the cells are also exposed to fibronectin or to a fibronectin fragment, wherein said fragment comprises a RGDS motif, a CS-1 motif and/or a heparin-binding domain. In some embodiments, the fibronectin fragment is Retronectin®.

In some embodiments, the fibronectin or fibronectin fragment is immobilized on an inner surface of a culture vessel or on beads.

In some embodiments, the CD34+ cells are cultured in a culture medium comprising TNF-α and/or an antagonist of the Aryl hydrocarbon/Dioxin receptor.

In some embodiments, the antagonist of the Aryl hydrocarbon/Dioxin receptor is StemRegenin 1 (SR1).

In some embodiments, the CD34+ cells are isolated from a human. In some embodiments, the CD34+ cells are isolated from an adult donor. In some embodiments, the CD34+ cells are isolated from cord blood.

In some embodiments, the T cell progenitors are autologous. In some embodiments, the T cell progenitors are allogenic.

The present invention further relates to T cell progenitors that comprise CD7+CD34− for treating, or for use in treating, aging, and in particular immunoaging, in a subject.

The present invention also relates to T cell progenitors that comprise CD7+CD34− for restoring, or for use in restoring, thymus function in a subject.

The present invention further relates to T cell progenitors that comprise CD7+CD34− for regenerating, or for use in regenerating the thymus of a subject.

In some embodiments, the T cell progenitors are for restoring or for use in restoring, polyclonal T cell production by the thymus of the subject.

In some embodiments, the subject is a human.

In some embodiments, the subject is of at least about 50 years in age, preferably of at least of about 60, 70, 80 or 90 years in age.

In some embodiments, the subject is affected or diagnosed with thymic atrophy or thymic involution.

In some embodiments, the CD7+CD34− cells comprise cells that are CD5− and/or CD1a−, preferably wherein at least about 50%, or at least about 60, 70, 80, 90 or 95% of the CD7+CD34− cells are CD5− and/or CD1a−.

In some embodiments, the T cell progenitors are genetically modified, such as, for example, to express an exogenous protein. For example, the exogenous protein may be a Chimeric Antigen Receptor (CAR) or a recombinant TCR.

In some embodiments, the T cell progenitors are transfected or transduced with an exogenous nucleic acid, encoding for example a Chimeric Antigen Receptor (CAR) or a recombinant TCR.

In some embodiments, the T cell progenitors express a cytokine, such as, for example, RANKL or Lymphotoxin A (LTA).

In some embodiments, the T cell progenitors are prepared by culturing CD34+ cells in the presence of an immobilized Notch ligand or of a fragment thereof, preferably wherein the Notch ligand is immobilized on the inner surface of the culture vessel or on beads.

In some embodiments, the cells are also exposed to fibronectin or to a fibronectin fragment, wherein said fragment comprises a RGDS motif, a CS-1 motif and/or a heparin-binding domain.

Thus, in some embodiments, the cells are also exposed to an immobilized Notch ligand or of a fragment thereof and to fibronectin or to a fibronectin fragment.

In some embodiments, the fibronectin or fibronectin fragment is immobilized on an inner surface of a culture vessel or on beads.

In some embodiments, the fibronectin fragment is Retronectin®.

In some embodiments, the CD34+ cells are cultured in a culture medium comprising TNF-α and/or an antagonist of the Aryl hydrocarbon/Dioxin receptor. In some embodiments, the antagonist of the Aryl hydrocarbon/Dioxin receptor is StemRegenin 1 (SR1).

In some embodiments, the CD34+ cells are isolated from a human. For example, the CD34+ cells may be isolated from an adult donor or from cord blood.

In some embodiments, the T cell progenitors are autologous.

In some embodiments, the T cell progenitors are allogenic.

### Definitions

In the present invention, the following terms have the following meanings:

“About” when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate to perform the disclose method.

“Cortical thymic epithelial cells (cTECs)”: refers to a component of the architecture of the thymic cortex. cTECs are known to have an essential role for commitment of early thymocyte precursors to the T cell lineage and for the positive selection of T cells.

“Medullary thymic epithelial cells (mTECs)”: refers to a component of the architecture of the thymic medulla. cTECs are known to have an essential role in thymocyte maturation and in the establishment of central tolerance (negative selection of T cells).

“Pharmaceutically acceptable excipient”: refers to an excipient that does not produce an adverse, allergic or other untoward reaction when administered to a mammal, preferably a human. It includes any and all solvents, dispersion media, coatings, antibacterial and antifungal agents, isotonic and absorption delaying agents and the like. A pharmaceutically acceptable carrier or excipient refers to a non-toxic solid, semi-solid or liquid filler, diluent, encapsulating material or formulation auxiliary of any type. For human administration, preparations should meet sterility, pyrogenicity, general safety and purity standards as required by the regulatory offices such as the FDA or EMA.

“Expressing”, “positive” or “+” and “not expressing”, “negative”, or “−” are refer to the expression level of a cell marker of interest, in that the expression level of the cell marker corresponding to “+” is high or intermediate or low (i.e., the cell marker is expressed or present at the cell surface), and the expression level of the cell marker corresponding to “−” is null (i.e., the cell marker is not expressed, or is absent, at the cell surface).

“Subject” refers to living organisms in which an immune response can be elicited (e.g., mammals, in particular human, primates, dogs, cats, horses, sheep and the like). In some embodiments, the subject is a human. In some embodiments, a subject may be a “patient”, i.e., a warm-blooded animal, preferably a human, who/which is awaiting the receipt of, or is receiving medical care or was/is/will be the object of a medical procedure or is monitored for the development of the targeted disease or condition. In some embodiments, the subject is a male. In another embodiment, the subject is a female.

“Therapeutically effective amount” refers to an amount of the cells or of the composition as described herein, effective to achieve a particular biological result. Thus, the terms “therapeutically effective amount” mean a level or amount of a composition or a number of cells that is aimed at, without causing significant negative or adverse side effects to the target, (1) delaying or preventing the onset of the targeted disease or condition; (2) slowing down or stopping the progression, aggravation, or deterioration of one or more symptoms of the targeted disease or condition; (3) bringing about ameliorations of the symptoms of the targeted disease or condition; (4) reducing the severity or incidence of the targeted disease or condition; or (5) curing the targeted disease or condition. A therapeutically effective amount may be administered prior to the onset of the targeted disease or condition, for a prophylactic or preventive action. Alternatively, or additionally, the therapeutically effective amount may be administered after initiation of the targeted disease or condition, for a therapeutic action.

“Transfection” or “transduction” refers to a process by which an exogenous nucleic acid is transferred or introduced into the host cell. A “transfected” or “transduced” cell is one which has been transfected, transformed or transduced with exogenous nucleic acid, and includes the primary subject cell and its progeny.

“Treating” refers to therapeutic treatment, and prophylactic or preventative measures; wherein the object is to prevent or slow down (lessen) the targeted disease or condition or to both therapeutic treatment and prophylactic or preventive measures. Those in need of treatment include those already with the condition as well as those prone to have the condition or those in whom the condition is to be prevented. A subject is successfully “treated” for a disease or condition if, after receiving a therapeutic amount of cells or compositions as described herein, the subject shows observable and/or measurable improvement in one or more of the following: increase in thymic size; increase of the size of the medulla, restoration of a normal thymic architecture, increase of the production of polyclonal T cells, relief to some extent of one or more of the symptoms associated with the specific condition; reduced morbidity and mortality, and/or improvement in quality of life issues. The above parameters for assessing successful treatment and improvement in the condition are readily measurable by routine procedures familiar to a physician.

### DETAILED DESCRIPTION

The present invention relates to a method of treating aging, and in particular immunoaging, in a subject, comprising the administration of T cell progenitors that comprise CD7+CD34− cells to the subject.

In some embodiments, immunoaging is a condition associated with aging, wherein the immune system of the subject is impaired (also called immunosenescence), and progressively loses its ability to protect the body against diseases or conditions, including, for example, infections or cancer. Immunoaging may for example be associated with an abnormal thymic architecture, leading to a decreased production of naïve T cells by the thymus, and to a decreased production of T cell progenitors by the bone marrow. Immunoaging may also be associated with inflammation (that may be called inflammaging). Inflammaging may in particular be associated with high levels of pro-inflammatory markers in cells and tissues, and with chronic inflammation.

The present invention also relates to a method of preventing, decreasing or slowing the progression of thymic atrophy or thymic involution comprising the administration of T cell progenitors that comprise CD7+CD34− cells to the subject.

Thymic atrophy or thymic involution may be caused by a disease (e.g., Di George syndrome), by aging or by treatments, such as, for example, chemotherapies or radiotherapies.

In some embodiments, thymic atrophy or involution is age-related. In some embodiments, thymic atrophy or involution is not age-related, and may be caused, for example, by a disease or by treatment with agents with thymic toxicity, such as, for example, a chemotherapy or a radiotherapy.

The present invention also relates to a method of restoring thymus function in a subject, comprising the administration of CD7+CD34− T cell progenitors to the subject. In some embodiments, the method comprises restoring thymopoiesis, i.e., the differentiation of thymocytes into mature T cells, the expansion of mature T cells, the positive selection of differentiating T cells and/or the establishment of central tolerance (including for example negative selection of T cells).

The present invention also relates to a method of regenerating the thymus of a subject, such as, for example, for restoring a normal function and/or architecture of the thymus, that was impaired by a disease or by aging or by treatments, such as, for example, chemotherapies or radiotherapies. In some embodiments, the method comprises restoring a normal architecture of the thymic epithelium, comprising a medulla and a cortex. For example, restoring a normal architecture of the thymic epithelium may be associated with an increased number and/or proportion of medullary thymic epithelial cells (mTEC).

The present invention also relates to a method for populating an empty thymus in a patient with severe immunodeficiency or in a patient lacking endogenous T cell progenitor production.

The present invention further relates to T cell progenitors that comprise CD7+CD34− cells for, or for use for treating aging, and in particular immunoaging, in a subject in need thereof.

The present invention further relates to a composition comprising or consisting essentially of T cell progenitors that comprise CD7+CD34− cells for, or for use for treating aging, and in particular immunoaging, in a subject in need thereof.

As used herein, the term “consisting essentially of”, with reference to a composition, pharmaceutical composition or medicament, means that the T cell progenitors are the only therapeutic agents or agents with a biologic activity within said composition, pharmaceutical composition or medicament.

The present invention further relates to the use of T cell progenitors that comprise CD7+CD34− cells for treating aging, and in particular immunoaging, in a subject in need thereof. The present invention further relates to the use of a composition comprising or consisting essentially of T cell progenitors that comprise CD7+CD34− cells for treating aging, and in particular immunoaging, in a subject in need thereof.

The present invention further relates to T cell progenitors that comprise CD7+CD34− cells for, or for use for, preventing, slowing or decreasing the progression of thymic atrophy or thymic involution. The present invention further relates to a composition comprising or consisting essentially of T cell progenitors that comprise CD7+CD34− cells for, or for use for, preventing, slowing or decreasing the progression of thymic atrophy or thymic involution.

The present invention further relates to the use of T cell progenitors that comprise CD7+CD34− cells for preventing, slowing or decreasing the progression of thymic atrophy or thymic involution. The present invention further relates to the use of a composition comprising or consisting essentially of T cell progenitors that comprise CD7+CD34− cells for preventing, slowing or decreasing the progression of thymic atrophy or thymic involution.

The present invention further relates to T cell progenitors that comprise CD7+CD34− cells for, or for use for, restoring thymus function in a subject. The present invention further relates to a composition comprising or consisting essentially of T cell progenitors that comprise CD7+CD34− cells for, or for use for, restoring thymus function in a subject.

The present invention further relates to the use of T cell progenitors that comprise CD7+CD34− cells for restoring thymus function in a subject. The present invention further relates to the use of a composition comprising or consisting essentially of T cell progenitors that comprise CD7+CD34− cells for restoring thymus function in a subject.

In some embodiments, T cell progenitors that comprise CD7+CD34− cells are for, or for use for, regenerating the thymus of a subject. In some embodiments, a composition comprising or consisting of T cell progenitors that comprise CD7+CD34− cells is for, or for use for, regenerating the thymus of a subject.

The present invention further relates to T cell progenitors that comprise CD7+CD34− cells for, or for use for, regenerating the thymus of a subject, such as, for example, for restoring a normal function and/or architecture of the thymus, that was impaired by a disease or by aging or by treatments, such as, for example, chemotherapies or radiotherapies. The present invention further relates to a composition comprising or consisting of T cell progenitors that comprise CD7+CD34− cells for, or for use for, regenerating the thymus of a subject, such as, for example, for restoring a normal function and/or architecture of the thymus, that was impaired by a disease or by aging or by treatments, such as, for example, chemotherapies or radiotherapies.

The present invention further relates to the use of T cell progenitors that comprise CD7+CD34− cells for regenerating the thymus of a subject. The present invention further relates to the use of a composition comprising or consisting essentially of T cell progenitors that comprise CD7+CD34− cells for regenerating the thymus of a subject.

The present invention further relates to T cell progenitors that comprise CD7+CD34− cells for, or for use for, populating an empty thymus in a patient with severe immunodeficiency or in a patient lacking endogenous T cell progenitor production. The present invention further relates to a composition comprising or consisting of T cell progenitors that comprise CD7+CD34− cells for, or for use for, populating an empty thymus in a patient with severe immunodeficiency or in a patient lacking endogenous T cell progenitor production.

The present invention further relates to the use of T cell progenitors that comprise CD7+CD34− cells for populating an empty thymus in a patient with severe immunodeficiency or in a patient lacking endogenous T cell progenitor production. The present invention further relates to the use of a composition comprising or consisting essentially of T cell progenitors that comprise CD7+CD34− cells for populating an empty thymus in a patient with severe immunodeficiency or in a patient lacking endogenous T cell progenitor production.

In some embodiments, the composition is a pharmaceutical composition, and further comprises a pharmaceutically acceptable excipient.

Examples of pharmaceutically acceptable excipients that may be used in the pharmaceutical composition of the invention include, but are not limited to, ion exchangers, alumina, aluminum stearate, lecithin, serum proteins, such as, for example, human serum albumin, buffer substances such as, for example, phosphates, glycine, sorbic acid, potassium sorbate, partial glyceride mixtures of saturated vegetable fatty acids, water, salts or electrolytes, such as, for example, protamine sulfate, disodium hydrogen phosphate, potassium hydrogen phosphate, sodium chloride, zinc salts, colloidal silica, magnesium trisilicate, polyvinyl pyrrolidone, cellulose-based substances (for example sodium carboxymethylcellulose), polyethylene glycol, polyacrylates, waxes, polyethylene-polyoxypropylene-block polymers, polyethylene glycol and wool fat.

In some embodiments, the composition is a medicament.

The present invention further relates to the use of T cell progenitors that comprise CD7+CD34− cells for the manufacture of a medicament for treating aging, and in particular immunoaging, in a subject in need thereof.

The present invention further relates to the use of T cell progenitors that comprise CD7+CD34− cells for the manufacture of a medicament for preventing, slowing or decreasing the progression of thymic atrophy or thymic involution.

The present invention further relates to the use of T cell progenitors that comprise CD7+CD34− cells for the manufacture of a medicament for restoring thymus function in a subject.

The present invention further relates to the use of T cell progenitors that comprise CD7+CD34− cells for the manufacture of a medicament for populating an empty thymus in a patient with severe immunodeficiency or in a patient lacking endogenous T cell progenitor production.

In some embodiments, a therapeutically effective amount of the T cell progenitors is administered (or is for administration) to a subject in need thereof.

In some embodiments, a therapeutically effective amount of the T cell progenitors is administered (or is for administration) to a subject in need thereof for regenerating the thymus of a subject. In some embodiments, a therapeutically effective amount of the T cell progenitors is administered (or is for administration) to a subject in need thereof for regenerating the thymus of a subject such as, for example, for restoring a normal function and/or architecture of the thymus, that was impaired by a disease or by aging or by treatments, such as, for example, chemotherapies or radiotherapies.

In some embodiments, a therapeutically effective amount of progenitor T cells ranges from about 1×10\hat{ }⁶ to about 100×10\hat{ }⁶ cells/kg body weight, preferably. from about 1×10\hat{ }⁶ to about 10×10\hat{ }⁶ cells/kg body weight.

In some embodiments, the subject is a human. In one embodiment, the subject is a male. In some embodiments, the subject is a female.

In some embodiments, the subject is an adult.

In some embodiments, the subject is of at least about 50 years in age, preferably of at least of about 60, 70, 80 or 90 years in age.

In some embodiments, the subject is a patient with severe immunodeficiency or a patient lacking endogenous T cell progenitor production. Examples of causes of severe immunodeficiencies include, but are not limited to, hereditary immune deficiency, immunodeficiency induced by chemotherapy (e.g., for treating leukemia), conditioning, graft with only stem cells, post-graft treatment for prophylaxis of GVH (graft-versus-host disease), age of patient, partial thymectomy, autoimmune disease and complications such as infections.

In some embodiments, the subject is affected, preferably is diagnosed, with thymic atrophy (also known as thymic involution). Thymic atrophy is associated with the loss of thymocytes, and eventually with the destruction of the thymic architecture. Thymic atrophy leads to a decrease in naïve T cells and limited T cell receptor diversity. Therefore, in some embodiments, the method of the invention is for restoring the polyclonal T cell production by the thymus of the subject. Thymic atrophy/involution is also associated with a decrease of thymic epithelial cells (TECs) i.e. a decrease of cortical thymic epithelial cell (cTEC) and medullary thymic epithelial cells (mTEC) and an accumulation of adipose tissue in the thymus.

In some embodiments, the subject is at risk of thymic atrophy. The risk of thymic atrophy may be related to a disease or to aging or to the toxicity of a treatment. In some embodiments, the subject is at risk of severe immunodeficiency.

In some embodiments, the subject is not affected, or is not diagnosed, with severe immunodeficiency. In some embodiments, the subject is not immunosuppressed.

In some embodiments, the subject is in need a hematopoietic stem cell transplant, such as for example, with CD34+ cells. In some embodiments, the subject received, is receiving or will receive a hematopoietic stem cell transplant, such as for example, with CD34+ cells.

In some embodiments, T cell progenitors administration is performed just prior to, just after or concomitantly with a hematopoietic stem cell transplant, such as for example, with CD34+ cells, in said subject.

In some embodiments, the subject doesn't need a hematopoietic stem cell transplant, such as for example, with CD34+ cells. In some embodiments, the subject has not received, is not receiving or will not receive a hematopoietic stem cell transplant, such as for example, with CD34+ cells.

In some embodiments, the T cell progenitors are genetically modified, such as, for example, to express an exogenous protein. In some embodiments, the T cell progenitors are transfected or transduced with an exogenous nucleic acid, wherein said exogenous nucleic acid may for example encode an exogenous protein.

In some embodiments, the T cell progenitors are genetically modified to silence a gene of interest, or to decrease the expression of a gene of interest by the cells.

In some embodiments, the T cell progenitors are genetically modified by a viral vector, a nucleic acid fragment, a plasmid or plasmidic RNA or DNA sequences. In some embodiments, the T cell progenitors are genetically modified using a system of gene editing, base editing or a system of prime-editing.

In some embodiments, the T cell progenitors comprise nucleic acid sequences encoding an element appropriate for gene editing. In some embodiments, the element appropriate for gene editing is a meganuclease, a zinc finger nuclease (ZFN), a transcription activator-like effector-based nuclease (TALEN), a CRISPR-Cas nuclease or a transposase (e.g., the Sleeping Beauty (SB)).

In some embodiments, the T cell progenitors are transfected or transduced with an exogenous nucleic acid encoding a Chimeric Antigen Receptor (CAR) or a recombinant TCR.

In some embodiments, the CD7+CD34− cells comprise cells that are CD5− and/or CD1a−, preferably wherein at least about 50%, or at least about 60, 70, 80, 90 or 95% of the CD7+CD34− cells are CD5− and/or CD1a−. In some embodiments, the CD7+CD34− cells comprise cells that are CD5−, preferably wherein at least about 50%, or at least about 60, 70, 80, 90 or 95% of the CD7+CD34− cells are CD5−. In some embodiments, the CD7+CD34− cells comprise cells that are CD1a−, preferably wherein at least about 50%, or at least about 60, 70, 80, 90 or 95% of the CD7+CD34− cells are CD1a−. In some embodiments, the CD7+CD34− cells comprise cells that are CD5−CD1a−, preferably wherein at least about 50%, or at least about 60, 70, 80, 90 or 95% of the CD7+CD34− cells are CD5−CD1a−.

In some embodiments, the T cell progenitors express at least one homing marker. In some embodiments, the T cell progenitors express at least one homing markers selected among the group comprising or consisting of CXCR4 (also known as CD184), CCR9, CD62L (also known L-selectin) and CCR7.

In some embodiments, the T cell progenitors comprise at least about 40, 41, 42, 43, 44, 45, 46, 47, 48, 49, 50, 51, 52, 53, 54, 55, 56, 57, 57, 59, 60, 61, 62, 63, 64, 65, 66, 67, 68, 69, or 70%, cells that express the marker CXCR4, preferably at least about 50%.

In some embodiments, the T cell progenitors comprise at least about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50%, cells that express the marker CCR9, preferably at least about 20%.

In some embodiments, the T cell progenitors comprise at least about 1, 2, 3, 4, 5, 6, 7, 8, 9, 10, 11, 12, 13, 14, 15, 16, 17, 18, 19, 20, 21, 22, 23, 24, or 25%, cells that express the marker CD62L, preferably at least about 10%.

In some embodiments, the T cell progenitors comprise at least about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35%, cells that express the marker CCR7, preferably at least about 15%.

In some embodiments, the T cell progenitors express at least one thymic rejuvenation/regeneration molecules. Examples of thymic rejuvenation/regeneration molecules include, but are not limited to RANKL and Lymphotoxin A (LTA).

In some embodiments, the T cell progenitors comprise at least about 1, 2, 3, 4, 5, 6, 7, 8, 9, 10, 11, 12, 13, 14, 15, 16, 17, 18, 19, 20, 21, 22, 23, 24, or 25%, cells that express the marker RANKL, preferably at least about 10%.

In some embodiments, the T cell progenitors comprise at least about 20, 25, 30, 35, 40, 45, 50, 55, 60 or 65%, cells that express the marker LTA, preferably at least about 30%.

In some embodiments, the T cell progenitors express a cytokine, in particular a cytokine involved in thymic regeneration, such as, for example, RANKL or LTA. In some embodiments, the T cell progenitors are genetically modified to express a cytokine, in particular a cytokine involved in thymic regeneration, such as, for example, RANKL or LTA.

In some embodiments, the T cells progenitors are autologous.

In some embodiments, the T cells progenitors are allogenic.

In some embodiments, the T cell progenitors are prepared by culturing CD34+ cells in the presence of an immobilized Notch ligand or of a fragment thereof. In some embodiments, the Notch ligand or the fragment thereof is immobilized on the inner surface of the culture vessel or on beads.

In some embodiments, the CD34+ cells are isolated from a human.

In some embodiments, the CD34+ cells are isolated from an adult donor. In some embodiments, the CD34+ cells are isolated from a bone marrow puncture or from peripheral blood from adult donors, which have been mobilized, such as, for example, using G-CSF.

In some embodiments, the CD34+ cells are isolated from umbilical cord blood.

Methods for isolating CD34+ cells are well known in the art and include, without limitation, methods using beads coated with an antibody recognizing CD34.

In some embodiments, the CD34+ cell population used in the method of the present invention is pure at least about 90, 91, 92, 93, 94, 95, 96, 97, 98 or 99%.

In some embodiments, the CD34+ cells are seeded at a concentration ranging from about 10⁶ to about 10⁷ cells/mL of culture medium.

In some embodiments, the culture vessel is selected conventional culture vessels comprising (but not limited to) culture plates from 6 to 96 wells, petri dishes, flasks, stirrer bottles, micro titer plates, test tubes, hollow fiber devices, cell foam and bags. The quantity of cells seeded may be adapted by one skilled in the art, according to the culture vessel used.

In some embodiments, the culture medium is adapted for the culture of CD34+ cells. Examples of culture medium adapted for culture of CD34+ cells include, but are not limited to, α-MEM, DMEM, RPMI 1640, IMDM, BME, McCoy's 5A, SFII (StemCell Technologies) media, Fischer's medium.

In some embodiments, the culture medium is feeder cell-free.

In some embodiments, the culture medium serum free. In one embodiment, the culture medium is supplemented with fetal bovine serum (FBS) or fetal calf serum (FCS), preferably with at least 15% or 20% v/v of FBS or FCS.

Notch proteins are transmembrane receptors that regulate the cellular response to a large number of environmental signals. In mammals, four Notch receptors (Notch 1-4) and five ligands (Delta-like-1, Delta-like-3, Delta-like-4, Jagged-1 and Jagged-2) have been described (Weinmaster Curr Opin Genet Dev 2000: 10: 363-369).

In some embodiments, the Notch ligand is Delta-like-4, preferably human Delta-like-4 (also known as DL-4, Uniprot accession number: Q9NR61, SEQ ID NO: 2), or a fragment thereof.

SEQ ID NO: 2
MAAASRSASGWALLLLVALWQQRAAGSGVFQLQLQEFINERGVLA
SGRPCEPGCRTFFRVCLKHFQAVVSPGPCTFGTVSTPVLGTNSFA
VRDDSSGGGRNPLQLPFNFTWPGTFSLIIEAWHAPGDDLRPEALP
PDALISKIAIQGSLAVGQNWLLDEQTSTLTRLRYSYRVICSDNYY
GDNCSRLCKKRNDHFGHYVCQPDGNLSCLPGWTGEYCQQPICLSG
CHEQNGYCSKPAECLCRPGWQGRLCNECIPHNGCRHGTCSTPWQC
TCDEGWGGLFCDQDLNYCTHHSPCKNGATCSNSGQRSYTCTCRPG
YTGVDCELELSECDSNPCRNGGSCKDQEDGYHCLCPPGYYGLHCE
HSTLSCADSPCFNGGSCRERNQGANYACECPPNFTGSNCEKKVDR
CTSNPCANGGQCLNRGPSRMCRCRPGFTGTYCELHVSDCARNPCA
HGGTCHDLENGLMCTCPAGFSGRRCEVRTSIDACASSPCFNRATC
YTDLSTDTFVCNCPYGFVGSRCEFPVGLPPSFPWVAVSLGVGLAV
LLVLLGMVAVAVRQLRLRRPDDGSREAMNNLSDFQKDNLIPAAQL
KNTNQKKELEVDCGLDKSNCGKQQNHTLDYNLAPGPLGRGTMPGK
FPHSDKSLGEKAPLRLHSEKPECRISAICSPRDSMYQSVCLISEE
RNECVIATEV

In some embodiments, the Notch ligand or the fragment thereof is the soluble domain of at least one Notch ligand. In some embodiments, the soluble domain of a Notch ligand represents the extracellular portion of said ligand.

In some embodiments, the Notch ligand or fragment thereof (preferably the soluble domain of the notch ligand) is fused to a protein allowing the Notch ligand to be immobilized on a support.

In some embodiments, the Notch ligand or fragment thereof (preferably the soluble domain of the notch ligand) is fused to a Fc region of an IgG protein, such as, for example, a human IgG protein. In some embodiments, the Notch ligand or fragment thereof (preferably the soluble domain of the notch ligand) is fused to a Fc region of an IgG2 protein, such as, for example, a human IgG2 protein (NCBI accession number: 4HAF_A, SEQ ID NO: 3).

SEQ ID NO: 3
VECPPCPAPPVAGPSVFLFPPKPKDTLMISRTPEVTCVVVDVSHE
DPEVQFNWYVDGVEVHNAKTKPREEQFNSTFRVVSVLTVVHQDWL
NGKEYKCKVSNKGLPAPIEKTISKTKGQPREPQVYTLPPSREEMT
KNQVSLTCLVKGFYPSDIAVEWESNGQPENNYKTTPPMLDSDGSF
FLYSKLTVDKSRWQQGNVFSCSVMHEALHNHYTQKSLSLSPGK

In some embodiments, the culture medium comprises DL-4 or a fragment thereof, preferably a fragment comprising or consisting of the soluble domain of the DL-4.

In some embodiments, the soluble domain of DL-4 comprises or consists of amino acids 1-526 of SEQ ID NO: 2. In another embodiment, the soluble domain of DL-4 comprises or consists of amino acids 1-525 of SEQ ID NO: 2. In another embodiment, the soluble domain of DL-4 comprises or consists of amino acids 1-524 of SEQ ID NO: 2.

In some embodiments, DL-4 or a soluble domain thereof is fused to the Fc receptor region of an IgG protein (such as, for example, a human IgG protein), in particular an IgG2 protein and preferably a human IgG2. An example of a protein comprising a soluble domain of DL-4 fused to the Fc receptor region of a human IgG2 protein is SEQ ID NO: 4.

SEQ ID NO: 4
MAAASRSASGWALLLLVALWQQRAAGSGVFQLQLQEFINERGVLA
SGRPCEPGCRTFFRVCLKHFQAVVSPGPCTFGTVSTPVLGTNSFA
VRDDSSGGGRNPLQLPFNFTWPGTFSLIIEAWHAPGDDLRPEALP
PDALISKIAIQGSLAVGQNWLLDEQTSTLTRLRYSYRVICSDNYY
GDNCSRLCKKRNDHFGHYVCQPDGNLSCLPGWTGEYCQQPICLSG
CHEQNGYCSKPAECLCRPGWQGRLCNECIPHNGCRHGTCSTPWQC
TCDEGWGGLFCDQDLNYCTHHSPCKNGATCSNSGQRSYTCTCRPG
YTGVDCELELSECDSNPCRNGGSCKDQEDGYHCLCPPGYYGLHCE
HSTLSCADSPCFNGGSCRERNQGANYACECPPNFTGSNCEKKVDR
CTSNPCANGGQCLNRGPSRMCRCRPGFTGTYCELHVSDCARNPCA
HGGTCHDLENGLMCTCPAGFSGRRCEVRTSIDACASSPCFNRATC
YTDLSTDTFVCNCPYGFVGSRCEFPVGLPPSTMVRSVECPPCPAP
PVAGPSVFLFPPKPKDTLMISRTPEVTCVVVDVSHEDPEVQFNWY
VDGMEVHNAKTKPREEQFNSTFRVVSVLTVVHQDWLNGKEYKCKV
SNKGLPAPIEKTISKTKGQPREPQVYTLPPSREEMTKNQVSLTCL
VKGFYPSDIAVEWESNGQPENNYKTTPPMLDSDGSFFLYSKLTVD
KSRWQQGNVFSCSVMHEALHNHYTQKSLSLSPGK

An example of a protein comprising a soluble domain of DL-4 fused to the Fc receptor region of an IgG1 protein (such as, for example, a human IgG1 protein) is a commercially available product (Sino Biologicals) comprising the extracellular domain (Met 1-Pro 524) of human DLL4 (full-length DLL4 accession number NP_061947.1) fused to the Fc region of human IgG1 at the C-terminus.

In some embodiments, the Notch ligand or fragment thereof is immobilized to the culture vessel used for the culture (i.e., bound to a solid support), although it is possible that certain elements may be found in solution. In some embodiments, the Notch ligand or fragment thereof is immobilized on the surface, preferably on the inner surface, of the culture vessel. Without willing to be bound to any theory, the Applicants suggest that immobilization of the Notch ligand or fragment thereof may stabilize it in order to facilitate interaction with the CD34+ cells and thus to allow activation of the Notch receptor of the CD34+ cells. In another embodiment, the Notch ligand or fragment thereof is immobilized on the surface of beads, preferably microbeads or such as polymer or magnetic beads (with a diameter generally comprised between 1 and 5 μm), present in the culture medium.

The binding of the Notch ligand or fragment thereof (e.g., to beads or to the surface of a culture vessel) may or may not be covalent. The binding of the Notch ligand or fragment thereof may be carried out non-covalently by allowing the Notch ligand or fragment thereof to be adsorbed onto the surface of the culture vessel or of beads. Methods for attaching a protein or peptide to beads or culture vessels are known in the art, and include, without limitation, fragment crystallizable (Fc) region of an immunoglobulin molecule (such as, e.g., human IgG).

A method to coat a culture vessel or beads with a Notch ligand or a fragment thereof is disclosed in WO2016/055396, which is incorporated herein by reference. In some embodiments, according to WO 2016/055396, around 75% of the Notch ligand or fragment thereof, in particular DL-4, will adhere to the culture vessel surface or to the beads surface when 5 μg/ml is used. In some embodiments, the composition used for coating a culture vessel or beads with a Notch ligand or fragment thereof comprises a concentration of the Notch ligand or fragment thereof higher or equal to 1.25 μg/ml and preferably ranging from about 2.5 and 5 μg/ml.

In some embodiments, the culture medium comprises fibronectin or a fibronectin fragment (fibronectin may have a sequence corresponding to the uniprot accession number: P02751, SEQ ID NO: 5). In some embodiments, the fibronectin fragment comprises or consists of an RGDS motif, a connecting segment 1 (CS-1) motif and/or a heparin binding domain. Preferably, the fibronectin fragment comprises or consists of an RGDS motif, a CS-1 motif and a heparin binding domain.

Fibronectin is a protein, which in its natural form is a v-shaped large dimer of 100 nm long and 460 kDa. The two monomers are connected by two disulfide bridges at their C-terminus. The term “fibronectin” or “fibronectin fragment” is understood to mean the natural fibronectin protein (i.e., any isoform produced by alternative splicing), but also a monomer of this protein, or a fragment of this protein (containing, when specified, the RGDS motif, CS-1 motif and heparin binding site).

An example of a fibronectin fragment which is particularly suitable for carrying out the process herein disclosed is Retronectin®. This protein corresponds to a fragment of a human fibronectin (CH-296 fragment, Kimizuka et al., J Biochem., 1991 Aug. 110 (2):284-91, Chono et al., J Biochem 2001 Sep 130 (3):331-4) and contains the cell-binding C domain (comprising the RGDS motif, the heparin-binding domain and the CS-1 motif). This protein is sold in particular by the companies Takara Bio Inc. (Shiga, Japan), Clinisciences (Nanterre, France, also called NovoNectin®) and Fisher scientific (Hampton, United-States).

In some embodiments, the fibronectin fragment is immobilized on an inner surface of a culture vessel or on beads.

The term “RGDS motif” is intended to designate any peptide or protein that contains the RGDS (SEQ ID NO: 6) pattern, so that it can bind integrin VLA-5. Such peptide or protein can be tested for its ability to bind VLA-5 integrin by methods known and reported in the art. RGDS motif binds to integrin VLA-5 (Very Late Antigen-5), which is a dimer composed of CD49e (alpha5) and CD29 (beta1).

Heparin-binding domains are known in the art and present in numerous proteins that bind to heparin. Their sequence is generally XBBXBX or XBBBXXBX (B=basic amino acid; X=hydropathic amino acid; Cardin and Weintraub, Arterioscler Thromb Vasc Biol. 1989; 9:21-32). Presence of such a heparin-binding domain is particularly favorable when the CD34+ cells are exposed to a viral (especially a retroviral) vector in order to transduce them and obtain T cell progenitors expressing a transgene.

A CS-1 motif is a 25 amino acids peptide (DELPQLVTLPHPNLHGPEILDVPST, SEQ ID NO: 7), as described by Wayner et al., 1989, J. Cell Biol. 109: 1321). The CS-1 motif binds to the VLA-4 (Very Late Antigen-4) receptor. VLA-4 is a dimer integrin, composed of CD49d (alpha 4) and CD29 (beta 1).

In some embodiments, the fibronectin or fibronectin fragment is immobilized (i.e., bound to a solid support). The binding of the fibronectin or fibronectin fragment (e.g., to beads or to the surface of a culture vessel) may or may not be covalent. In some embodiments, the fibronectin or fibronectin fragment is immobilized to the inner surface of the culture vessel (although it is possible that certain elements may be found in solution). In another embodiment, the fibronectin or fibronectin fragment is immobilized on the surface of beads, preferably microbeads or such as polymer or magnetic beads (with a diameter generally comprised between 1 and 5 μm). In some embodiments, the Notch ligand or fragment thereof and the fibronectin or fragment thereof are immobilized on the same beads. In another embodiment, the Notch ligand or fragment thereof and the fibronectin or fragment thereof are immobilized on the distinct beads.

In some embodiments, immobilization of the fibronectin or fibronectin fragment is carried out non-covalently by allowing the fibronectin or fragment thereof to be adsorbed onto the inner surface of the culture vessel or onto the surface of beads. Methods for attaching a protein or peptide to beads or to the surface of a culture vessel are known in the art and are listed hereinabove.

A method to coat a culture vessel or beads with fibronectin or a fragment thereof is disclosed in WO2016/055396. In some embodiments, the composition used for coating a culture vessel or beads with fibronectin or a fragment thereof comprises a concentration of fibronectin or a fragment thereof ranging from 10 and 100 μg/ml, preferably of about 25 μg/ml.

In some embodiments, the culture medium comprises at least 1, 2 or 3 (such as, for example, 1, 2 or 3) cytokines selected from the group comprising or consisting of SCF (stem cell factor), Flt3-L (Flt3 ligand), and IL-7. In some embodiments, the culture medium comprises at least 1, 2 or 3 (such as, for example, 1, 2 or 3) cytokines selected from the group comprising or consisting of human SCF, human Flt3-L, and human IL-7.

In some embodiments, the culture medium comprises at least 1, 2, 3 or 4 (such as, for example, 1, 2, 3, or 4) cytokines selected from the group comprising or consisting of SCF (stem cell factor), Flt3-L (Flt3 ligand), TPO (thrombopoietin) and IL-7 (interleukin 7). In some embodiments, the culture medium comprises at least 1, 2, 3 or 4 (such as, for example, 1, 2, 3, or 4) cytokines selected from the group comprising or consisting of human SCF, human Flt3-L, human TPO and human IL-7.

hSCF, hFlt3-L, hTPO and hIL-7 are provided, for example, by Peprotech.

In some embodiments, the concentration of SCF, preferably of hSCF ranges from about 2 to about 300 ng/mL, preferably from about 40 to about 300 ng/mL or from about 40 ng/mL to about 200 ng/mL and more preferably is of about 100 ng/mL.

In some embodiments, the concentration of Flt3-L, preferably of hFlt3-L ranges from about 2 to about 300 ng/mL, preferably from about 40 to about 300 ng/mL or from about 40 ng/mL to about 200 ng/mL and more preferably is of about 100 ng/mL.

In some embodiments, the concentration of TPO, preferably of hTPO ranges from about 2 to about 300 ng/mL, preferably from about 40 to about 300 ng/mL or from about 40 ng/mL to about 200 ng/mL and more preferably is of about 100 ng/mL.

In some embodiments, the concentration of IL-7, preferably of hIL-7 ranges from about 2 to about 300 ng/mL, preferably from about 40 to about 300 ng/mL or from about 40 ng/mL to about 200 ng/mL and more preferably is of about 100 ng/mL.

In some embodiments, the medium comprises IL-3, preferably human IL-3. In some embodiments, the medium does not comprise IL-3.

In some embodiments, CD34+ cells are cultured in a culture medium comprising TNF-α and/or an antagonist of the Aryl hydrocarbon/Dioxin receptor.

In some embodiments, CD34+ cells are cultured in a culture medium comprising a Notch ligand or a fragment thereof, TNF-α and fibronectin or a fragment thereof. In some embodiments, CD34+ cells are cultured in a culture medium comprising a Notch ligand or a fragment thereof, an antagonist of the Aryl hydrocarbon/Dioxin receptor and fibronectin or a fragment thereof. In some embodiments, CD34+ cells are cultured in a culture medium comprising a Notch ligand or a fragment thereof, TNF-α, an antagonist of the Aryl hydrocarbon/Dioxin receptor and fibronectin or a fragment thereof.

In some embodiments, TNF-α is human TNF-α, having for example the sequence of SEQ ID NO: 1 (Uniprot accession number: P01375).

SEQ ID NO: 1
MSTESMIRDVELAEEALPKKTGGPQGSRRCLFLSLFSFLIVAGAT
TLFCLLHFGVIGPQREEFPRDLSLISPLAQAVRSSSRTPSDKPVA
HVVANPQAEGQLQWLNRRANALLANGVELRDNQLVVPSEGLYLIY
SQVLFKGQGCPSTHVLLTHTISRIAVSYQTKVNLLSAIKSPCQRE
TPEGAEAKPWYEPIYLGGVFQLEKGDRLSAEINRPDYLDFAESGQ
VYFGIIAL

TNF-α is primarily produced as a type II transmembrane protein arranged in stable homodimers, each monomer comprising 233 amino acids in human. In human, the soluble part of human TNF-α is composed of amino acid 77 to 233 of SEQ ID NO: 1.

In some embodiments, culture medium comprises full-length TNF-α or a soluble fragment thereof.

In some embodiments, TNF-α or the fragment thereof is added from day 0 of culture. In some embodiments, TNF-α or the fragment thereof is present in the culture medium since day 0 and during at least about 1, 2, 3, 4, 5, 6 or 7 days. In some embodiments, TNF-α or the fragment thereof is present in the culture medium from day 0 to the end of the culture.

In some embodiments, TNF-α or the fragment thereof is used at a concentration ranging from about 10 to about 300 ng/mL, such as, for example, of at least about 10, 20, 30, 40, 50, 100, 200 or 300 ng/mL. In some embodiments, TNF-α or the fragment thereof is used at a concentration of about 10 ng/mL. However, other concentrations such as about 5, 10, 20 or 50 ng/mL are also suitable.

In some embodiments, the antagonist of the Aryl hydrocarbon/Dioxin receptor is StemRegenin 1 (SR1, 4-(2-(2-(Benzo[b]thiophen-3-yl)-9-isopropyl-9H-purin-6-ylamino)ethyl)phenol, CAS 1227633-49-10).

In some embodiments, the antagonist of the Aryl hydrocarbon/Dioxin receptor is present in the culture medium from day 0 of the culture.

In some embodiments, the antagonist of the Aryl hydrocarbon/Dioxin receptor is added to the medium culture at a concentration ranging from about 1 ng/ml to about 300 ng/ml and preferably higher or equal to 1 ng/ml, or higher or equal to 3 ng/ml, or higher or equal to 10 ng/ml, and preferably lower than 200 ng/ml, or 150 ng/ml and generally between 3 ng/ml and 100 ng/ml.

In some embodiments, the CD34+ cells are cultured for at most 10 days, preferably for 3 to 7 days.

### BRIEF DESCRIPTION OF THE DRAWINGS

FIG. 1 is a plot showing the expression of CD34 and CD7 by HTLP cells obtained after culturing CD34+ cells in the presence IL-7, Flt3-L, SCF, TPO, TNFα and in presence of immobilized DLL4/RetroNectin for 7 days.

FIGS. 2A and 2B are a combination of UMAP plots showing the expression of homing markers: CXCR4, CCR9, CD62L and CCR7 by HTLP cells obtained after culturing CD34+ cells in the presence IL-7, Flt3-L, SCF, TPO, TNFα and in presence of immobilized DLL4/RetroNectin for 7 days. The surrounded portion of the plot indicates the cells expressing the marker of interest (i.e. CXCR4, CCR9, CD62L or CCR7) (A) FIG. 2A is a combination of UMAP plot showing the expression CXCR4, CCR9 and CD62L transcripts characterized with single cell RNAseq (scRNAseq). Darker is gray, higher is the expression of the gene. (B) FIG. 2B is a combination of UMAP plot showing the expression of CXCR4, CCR9 CD62L and CCR7 at protein level characterized with mass cytometry. Darker is gray, lower is the amount of protein.

FIG. 3 is a combination of UMAP plots showing the expression of thymic rejuvenation molecules: Lymphotoxin a (LTA) and RANKL, by HTLP cells obtained after culturing CD34+ cells in the presence IL-7, Flt3-L, SCF, TPO, TNFα and in presence of immobilized DLL4/RetroNectin for 7 days. The surrounded portion of the plot indicates the cells expressing the protein of interest (i.e. Lymphotoxin a (LTA) or RANKL). Darker is gray, lower is the amount of protein

FIGS. 4A and 4B are a combination of confocal microscopy and histology images showing HTLP cells (CD45+, light gray) infiltrating in thymic epithelial stroma composed of cortical thymic epithelial cells (cTEC) (Keratin 8+) and medullary epithelial cells (mTEC) (Keratin 14+). FIG. 4A is a combination of confocal microscopy and histology images showing HTLP cells (CD45+, light gray) infiltration at two weeks. Darker is the gray, lower is the colonization by human CD45+ cells. FIG. 4B is a combination of confocal microscopy and histology images showing HTLP cells (CD45+, light gray) infiltration at ten weeks. Light gray zones correspond to CD45+ cells (upper panel). Surrounded surface correspond to medullary islands composed of keratin 14+ TEC (mTEC) and clusters of CD45+(lower panel).

FIG. 5 is a graph showing the ratio of medullary TEC to cortical TEC (mTEC/cTEC) after HTLP cells engraftment or after CD34+ HSPCs engraftment.

FIGS. 6A-6C are a combination of schematic representation and Flow cytometry plots showing the reconstitution of complete T-cell compartment by HTLP cells after administration in adult immunodeficient mice after irradiation. FIG. 6A is a schematic representation of the protocol of HTLP cells injection in adult immunodeficient mice after irradiation. FIG. 6B is a flow cytometry plot showing the expression of human CD4 and human CD8 on cells present in the thymus of mice 12 weeks post injection. FIG. 6C is a flow cytometry plot showing the expression of human CD3 and human TCRαβ on cells present in the thymus of mice 12 weeks post injection.

FIGS. 7A and 7B are a combination of schematic representation and histogram showing the engraftment of human cells in the thymus of aged irradiated mice after HTLP cells administration. FIG. 7A is a schematic representation of the protocol of HTLP cells injection in aged mice after irradiation. FIG. 7B is a histogram showing the percentage of human CD45 cells in the thymus of aged irradiated mice injected with CD34+ cells or co-injected with CD34+ cells and HTLP cells.

### EXAMPLES

The present invention is further illustrated by the following examples.

### Example 1

### Materials and Methods

### Generation and Characterization of Human T Lymphoid Progenitor

CD34+ HSPCs isolated from cord blood (CB) were cultured for 7 days in α-MEM supplemented with 20% defined fetal bovine serum and the following human cytokines: TL-7, Flt3-L, SCF, TPO, TNFα on DLL4/RetroNectin-coated culture plate. Generated Human T Lymphoid progenitor (HTLP, also called T cell progenitors) cells were then analyzed by single-cell RNAseq (scRNAseq) and mass cytometry.

### In Vivo Characterization of Human T Lymphoid Progenitor

Generated HTLP cells were intra hepatically injected in neonatal NSG (NOD.Cg-Prkdc^{scid} Il2rg^{tm1Wjl}/SzJ) mice (between 1 and 4-day-old) at the dose of 1M cells per recipient. T cell engraftment and thymic rejuvenation was assessed by flow cytometry and histology (confocal).

### Results

### Generated HTLP Express Thymus Homing Markers and Thymic Regeneration Markers

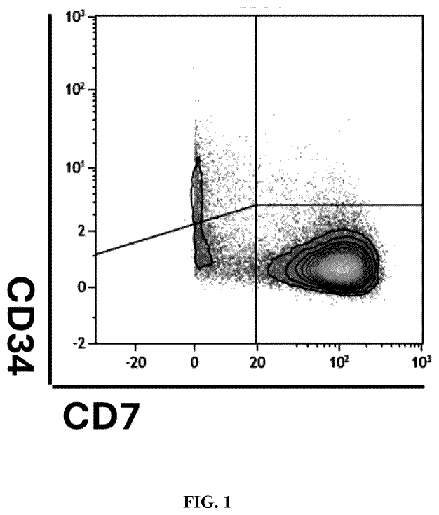

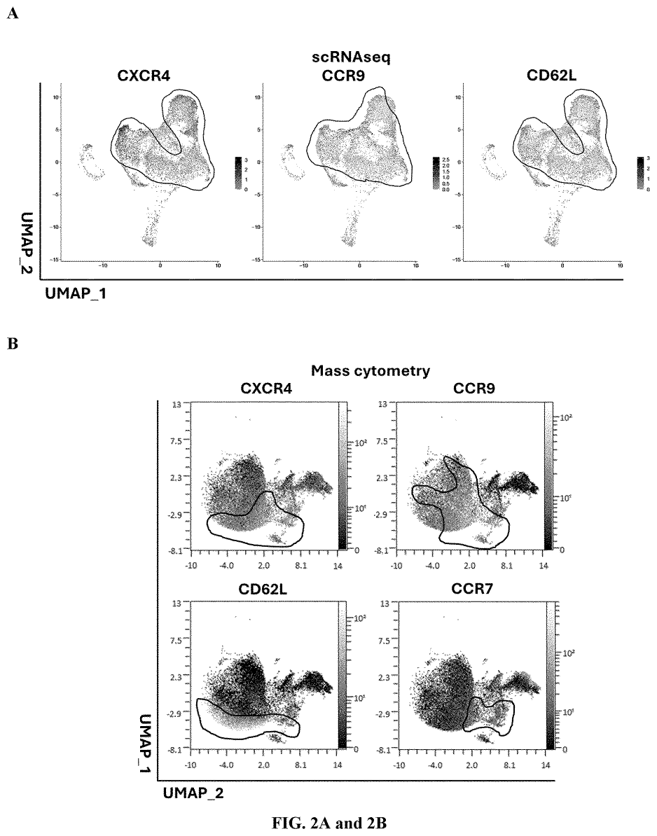

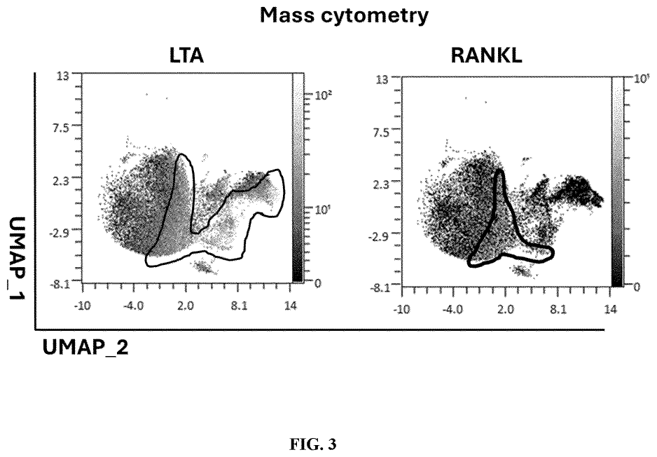

Generated HTLP cells are characterized as CD34-CD7+ cells (see FIG. 1). As shown in FIGS. 2A and 2B, said cells express various chemokine receptors such as CXCR4, CCR9 and CCR7 (the surrounded portion of the plot indicates the cells expressing the gene or protein of interest) know to be essential for thymus entry. These progenitors are also defined by the expression of adhesion molecules including CD62L which is involved in homing to lymphoid organs and has proven to correlate with thymic engraftment in progenitors. As shown in FIG. 3, HTLP cells also strongly express Lymphotoxin a (LTA), that plays a critical role in the physiological regulation of lymphoid tissues and thymic epithelium recovery. Generated HTLP cells also express RANKL that act on thymic development and regeneration by specifically promoting mature medullary TECs (thymic epithelial cells) maturation.

Taken together, the results suggest that Generated HTLP cells could be able to migrate into the thymus and could play a role in thymic recovery.

Thymic Medullary Epithelium is Enlarged with HTLP Graft

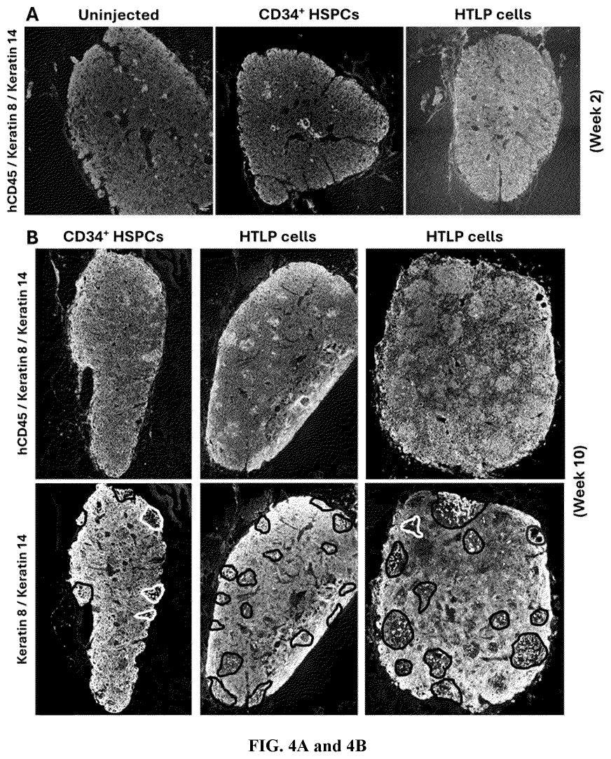

The thymic epithelial stroma of NSG mice is mostly composed of cortical thymic epithelial cell (cTEC) (Keratin 8+) and contains only few medullary thymic epithelial cells (mTEC). Following the successful seeding with HTLP cells, the thymus of NSG recipients display a larger thymus that contains individual medullary islands composed of keratin 14+ TEC and clusters of CD45+ progeny of the transplanted progenitors (see FIGS. 4 A and 4B). Indeed, in FIG. 4B showing the thymi at week 10, larger is the surrounded surface, higher is the proportion of medulla (surrounded zones, lower panel) and higher is the colonization by human CD45+ cells (light gray, upper panel). These morphological features are characteristic of an effective thymopoiesis and typically absent in non-grafted mice and delayed/lesser in the one injected with CD34+ HSPCs.

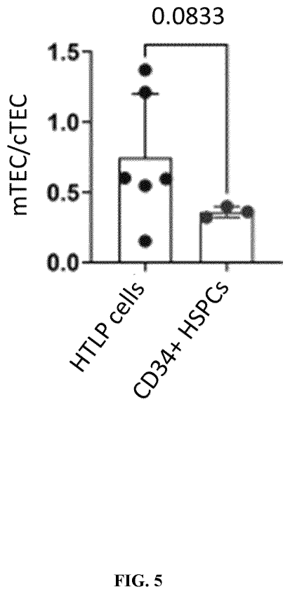

As shown in FIG. 5, following the engraftment with HTLP cells tends in NGS recipients to increase the ratio of medullary TEC to cortical TEC (mTEC/cTEC), correlating with thymic regeneration and progression of thymocytes to a developmental stage located in the medulla. This result highlights the superior effect of HTLP cells on the intrathymic architecture and composition of the epithelial stroma and the faster kinetic in thymic reconstitution following the engraftment of these progenitors.

Taken together, these results reveal the regeneration attributes of generated HTLP cells together with their engraftment ability. The presence of medullary islands reveal also the ability of HTLP cells to regenerate thymic epithelium.

### Example 2

### Materials and Methods

### Transplantation of HTLP Cells in Adult Immunodeficient Mice

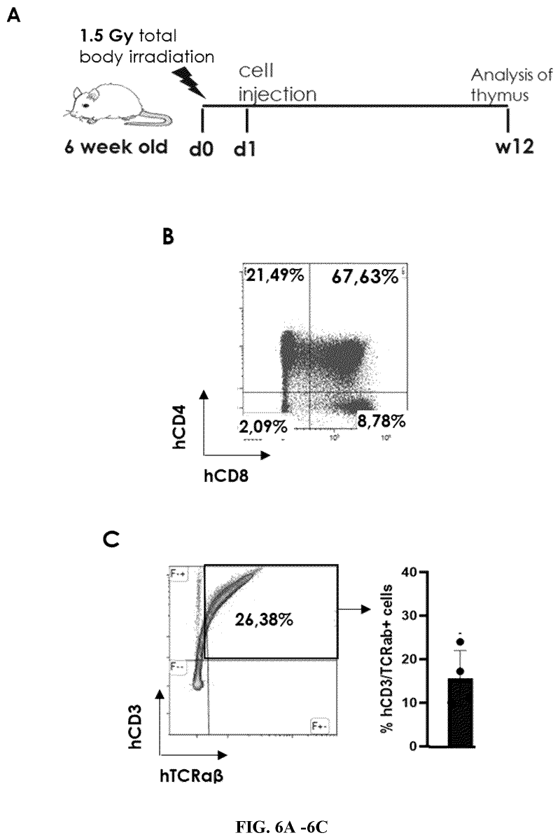

6-8 weeks old NSG ((NOD.Cg-Prkd^{scid} Il2rg^{tm1Wjl}/SzJ) mice have been irradiated at 1.5 Gy at day 0. Twenty-four hours after irradiation, mice have received intravenously a single dose of HTLP cells derived from mobilized Peripheral Blood (mPB) equivalent to 6.25×10⁶ CD7+ expressing cells (FIG. 6A). At week 12 post injection, the thymy have been harvested and analyzed with flow cytometry to characterize the thymocytes by different anti human antibodies (hCD45, hCD4, hCD8, hCD3, hTCRab) (BD Biosciences) (FIGS. 6B and 6C).

### Results

As shown in FIG. 6B, 12 weeks after the injection of HTLP cells, human CD4⁺ CD8⁺ double positive (DP), CD4⁺ simple positive (CD4 SP) and CD8⁺ simple positive (CD8 SP) thymocytes have been characterized in the thymus of adult immunodeficient mice. The generation of said different population of cells demonstrates an active and efficient human thymopoiesis. As shown in FIG. 6C, human cells are also expressing CD3 and TCRab in the thymus.

Taken together, these results reveal the reconstitution of complete T-cell compartment by HTLP cells administration in adult immunodeficient mice after irradiation. These results reveal the ability to HTLP to restore thymic function.

### Example 3

### Materials and Methods

### Co-Transplantation of Human Hematopoietic Stem Cells (CD34+) and HTLP Cells in Aged Immunodeficient Mice

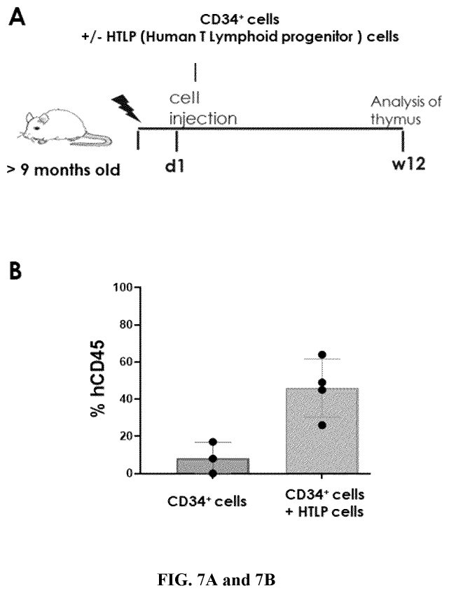

NSG mice (more than 9 months old) have been irradiated at 1.5 Gy at day 0. (FIG. 7A) Twenty-four hours after irradiation, mice have been co-injected intravenously by 0.3×10⁶ CD34⁺ cells and HTLP derived from mPB equivalent to 1×10⁶ CD7⁺ expressing cells. One control group has received only 0.3×10⁶ CD34⁺ cells without HTLP cells. At week 12 post injection, the thymy have been harvested and the chimerism of the human cells in the thymus has been analyzed by flow cytometry using hCD45 and mCD45 antibodies (BD Biosciences).

### Results

As shown in FIG. 7B, 12 weeks after injection the percentage of human CD45+ cells are significantly increased in the thymy of co-injected mice in comparison of the thymy of mice receiving only CD34+ cells (Respectively 46% and 7.6%). Said results demonstrate that the engraftment and chimerism of human cells is significantly increased in co-injected aged mice. Results demonstrate also an effect of HTLP cells on the thymus and human cell engraftment.

These results suggest that HTLP cells contribute to the reconstitution of T-cell compartment in aged subject.

1. T cell progenitors that comprise CD7+CD34− cells for use in treating aging, and preferably immunoaging in a subject.

2. T cell progenitors that comprise CD7+CD34− cells for use in restoring thymus function in a subject.

3. T cell progenitors that comprise CD7+CD34− cells for use in regenerating the thymus of a subject.

4. The T cell progenitors for use according to any one of claims 1 to 3, for use in restoring polyclonal T cell production by the thymus of the subject.

5. The T cell progenitors for use according to any one of claims 1 to 4, wherein the subject is a human.

6. The T cell progenitors for use according to any one of claims 1 to 5, wherein the subject is of at least about 50 years in age, preferably of at least of about 60, 70, 80 or 90 years in age.

7. The T cell progenitors for use according to any one of claims 1 to 6, wherein the subject is affected or diagnosed with thymic atrophy or thymic involution.

8. The T cell progenitors for use according to any one of claims 1 to 7, wherein the CD7+CD34− cells comprise cells that are CD5− and/or CD1a−, preferably wherein at least about 50%, or at least about 60, 70 or 80% of the CD7+CD34− cells are CD5− and/or CD1a−.

9. The T cell progenitors for use according to any one of claims 1 to 8, wherein the T cell progenitors are genetically modified to express an exogenous protein, preferably wherein said exogenous protein is a Chimeric Antigen Receptor (CAR) or a recombinant TCR.

10. The T cell progenitors for use according to any one of claims 1 to 9, wherein the T cell progenitors express a cytokine, preferably, RANKL or Lymphotoxin A (LTA).

11. The T cell progenitors for use according to any one of claims 1 to 10, wherein the T cell progenitors are prepared by culturing CD34+ cells in the presence of an immobilized Notch ligand or of a fragment thereof, and preferably of fibronectin or a fragment thereof, more preferably wherein the fibronectin fragment comprises a RGDS motif, a CS-1 motif and/or a heparin-binding domain.

12. The T cell progenitors for use according to claim 11, wherein the CD34+ cells are cultured in a culture medium comprising TNF-α and/or an antagonist of the Aryl hydrocarbon/Dioxin receptor, preferably wherein the antagonist of the Aryl hydrocarbon/Dioxin receptor is SR1.

13. The T cell progenitors for use according to any one of claims 11 to 12, wherein the CD34+ cells are isolated from a human, preferably from an adult donor or from cord blood.

14. The T cell progenitors for use according to any one of claims 1 to 13, wherein the T cell progenitors are autologous.

15. The T cell progenitors for use according to any one of claims 1 to 13, wherein the T cell progenitors are allogenic.


SEQUENCE LISTING (7 sequences)

SEQ ID NO: 1 - protein, 233 aa, synthetic construct
  REGION 1..233: note = TNF-alpha
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 2 - protein, 685 aa, synthetic construct
  REGION 1..685: note = Delta-like-4
MAAASRSASGWALLLLVALWQQRAAGSGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPSFPWVAVSLGVGLAVLLVLLGMVAVAVRQLRLRRPDDGSREAMNNLSDFQKDNLIPAAQLKNTNQKKELEVDCGLDKSNCGKQQNHTLDYNLAPGPLGRGTMPGKFPHSDKSLGEKAPLRLHSEKPECRISAICSPRDSMYQSVCLISEERNECVIATEV

SEQ ID NO: 3 - protein, 223 aa, synthetic construct
  REGION 1..223: note = IgG2Fc
VECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 4 - protein, 754 aa, synthetic construct
  REGION 1..754: note = Delta-like-4-IgG2Fc
MAAASRSASGWALLLLVALWQQRAAGSGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPSTMVRSVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 5 - protein, 2477 aa, synthetic construct
  REGION 1..2477: note = fibronectin
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVSWVSASDTVSGFRVEYELSEEGDEPQYLDLPSTATSVNIPDLLPGRKYIVNVYQISEDGEQSLILSTSQTTAPDAPPDTTVDQVDDTSIVVRWSRPQAPITGYRIVYSPSVEGSSTELNLPETANSVTLSDLQPGVQYNITIYAVEENQESTPVVIQQETTGTPRSDTVPSPRDLQFVEVTDVKVTIMWTPPESAVTGYRVDVIPVNLPGEHGQRLPISRNTFAEVTGLSPGVTYYFKVFAVSHGRESKPLTAQQTTKLDAPTNLQFVNETDSTVLVRWTPPRAQITGYRLTVGLTRRGQPRQYNVGPSVSKYPLRNLQPASEYTVSLVAIKGNQESPKATGVFTTLQPGSSIPPYNTEVTETTIVITWTPAPRIGFKLGVRPSQGGEAPREVTSDSGSIVVSGLTPGVEYVYTIQVLRDGQERDAPIVNKVVTPLSPPTNLHLEANPDTGVLTVSWERSTTPDITGYRITTTPTNGQQGNSLEEVVHADQSSCTFDNLSPGLEYNVSVYTVKDDKESVPISDTIIPEVPQLTDLSFVDITDSSIGLRWTPLNSSTIIGYRITVVAAGEGIPIFEDFVDSSVGYYTVTGLEPGIDYDISVITLINGGESAPTTLTQQTAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTNIDRPKGLAFTDVDVDSIKIAWESPQGQVSRYRVTYSSPEDGIHELFPAPDGEEDTAELQGLRPGSEYTVSVVALHDDMESQPLIGTQSTAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPSTVQKTPFVTHPGYDTGNGIQLPGTSGQQPSVGQQMIFEEHGFRRTTPPTTATPIRHRPRPYPPNVGEEIQIGHIPREDVDYHLYPHGPGLNPNASTGQEALSQTTISWAPFQDTSEYIISCHPVGTDEEPLQFRVPGTSTSATLTGLTRGATYNVIVEALKDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 6 - protein, 4 aa, synthetic construct
  REGION 1..4: note = RGDS motif
RGDS

SEQ ID NO: 7 - protein, 25 aa, synthetic construct
  REGION 1..25: note = CS-1 motif
DELPQLVTLPHPNLHGPEILDVPST